(19) United States
(12) Patent Application Publication
(10) Pub. No.: US 2005/0228482 A1
(43) Pub. Date: Oct. 13, 2005

(54) Stent covered by a layer having a layer opening

(76) Inventors: William Herzog, Baltimore, MD (US); Yi-Ju Zhao, Ellicot City, MD (US); Miriam Lazur, Walnut Creek, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 10/950,307

(22) Filed: Sep. 23, 2004

Related U.S. Application Data
(60) Provisional application No. 60/505,872, filed on Sep. 26, 2003.

(51) Int. Cl. A61F 2/06

(52) U.S. Cl. 623/1.15; 623/1.42

(57) ABSTRACT

A drug eluting stent has a frame with at least one frame opening, and the frame is covered with an outer continuous layer and/or an inner continuous layer. The outer and/or inner layers further include a layer opening that at least partially overlaps with the frame opening to form a continuous opening having a size that allows endothelialization when the stent is implanted into a blood vessel.

This application claims the benefit of U.S. provisional patent application No. 60/505,872, which was filed Sep. 26, 2003, and which is incorporated by reference herein.

### FIELD OF THE INVENTION

The field of the invention is implantable devices, and particularly stents for interventional cardiology, urology, and nephrology.

### BACKGROUND OF THE INVENTION

Stents are commonly used to maintain the diameter of a lumen in a biological vessel that has been previously obstructed or even blocked. However, an implanted stent is often recognized by the body as a foreign object, and restenosis may occur due to platelet deposition, thrombosis, and other mechanisms.

To prevent restenosis, numerous compositions and methods are known that relate to drug elution from a stent, and particularly from braided stents. For example, as described by Pinchuk in U.S. Pat. No. 5,092,877 or Ding et al. in U.S. Pat. No. 5,837,313, a stent is permanently coated with a drug delivery agent. On the other hand, a stent may also be coated with a biodegradable or absorbable polymer that releases the drug as a function of the degradation/absorption as taught by Tang et al. in U.S. Pat. No. 4,916,193, and MacGregor in U.S. Pat. No. 4,994,071. Further known stent coatings include those described by Sanders Millare et. al. in U.S. Pat. No. 6,540,776, in which a drug eluting sheath is coupled to a stent, and wherein the sheath has interstices to allow blood to seep through the sheath in the direction of the pattern created.

In still further known drug release stents, the stent material acts as a carrier for the drug as described in U.S. Pat. App. No. 2004/0143322 to Litvack et al. Here, the stent frame has bores from which the drugs are eluted. Alternatively, as described in U.S. Pat. No. 5,163,952 to Froix, a polymeric stent acts as the carrier from which the drug is eluted. While many coatings have a somewhat linear drug release characteristic independent of the environment in which they are placed, other coatings, as described by Sahatjian in U.S. Pat. No. 5,304,121 provide specific drug release in response to pressure.

Unfortunately, while many drug eluting stents tend to reduce the incidence and/or severity of platelet aggregation and smooth muscle cell growth, the stent coating in all or almost all of such stents will inhibit growth of endothelial cells into the stent. On the other hand, where only the filaments of a braided stent are covered with a drug eluting composition, the amount of drug available is typically insufficient to provide a substantial therapeutic effect. Consequently, known drug eluting stents fail to promote integration of the implant by endothelialization.

Thus, while there are numerous configurations and methods for drug eluting stents known in the art, all or almost all of them suffer from various problems. Therefore, there is still a need for improved drug eluting stents, and especially for those that promote endothelialization.

### SUMMARY OF THE INVENTION

The present invention is directed to a stent that includes a frame with at least one frame opening, wherein the frame is covered with an outer continuous layer and/or an inner continuous layer. The outer and/or inner layers in contemplated stents further include a layer opening that at least partially overlaps with the frame opening to form a continuous opening having a size that allows endothelialization when the stent in implanted into a blood vessel. Especially preferred stents also include a pharmaceutical compound that releases a pharmaceutically active agent to the site of implantation.

Thus, in one aspect of the inventive subject matter, a stent comprises a cylindrical frame with an outer surface, an inner surface, and a plurality of frame openings extending from the outer surface to the inner surface. A continuous inner layer with an inner layer opening is coupled to the inner surface, and/or a continuous outer layer having an outer layer opening is coupled to the outer surface of the frame surface, wherein the frame openings, inner layer opening, and/or the outer layer opening coincide such that an aperture is formed having a size sufficient to allow endothelialization. It is still further preferred that the a pharmaceutically active agent is associated with the inner layer, the inner surface, another frame opening, the outer surface, and/or the outer layer.

It is contemplated that the pharmaceutically active agent can be disposed onto the inner surface and the outer surface, within another frame opening, and/or that the pharmaceutically active agent is covered by the outer layer. It is also contemplated that the pharmaceutically active agent can be disposed in a polymeric matrix that is coated onto the cylindrical frame and at least partially covered by the continuous outer layer. Especially preferred pharmaceutically active agents modulate vascular tone, enhance macrophage-mediated microbial killing, and/or inhibit neutrophil adhesion, platelet adhesion, platelet aggregation, and/or smooth muscle cell proliferation.

While inner and outer layers can be fabricated from a wide variety of materials, it is generally preferred that at least one of the layers is fabricated from a material that allows diffusion of a gas and that prevents diffusion of a liquid (e.g., fluorinated synthetic polymer). Similarly, the cylindrical frame may be fabricated from numerous materials. However, it is generally preferred that the cylindrical frame comprises a metal. Thus, the cylindrical frame, the inner layer, and the outer layer in preferred stents are fabricated from a material that allows radial expansion of the stent.

Viewed from another perspective, contemplated stents can be composite stents having a cylindrical multilayer structure comprising an inner layer at least partially surrounded by a frame layer that is at least partially enclosed by an outer layer, and that further comprises an opening extending through the multilayer structure, wherein the opening has a size sufficient to allow endothelialization when the composite stent in implanted into a blood vessel.

Particularly preferred composite stents include a pharmaceutical agent that provides a pharmaceutically active compound to the site of implantation of the composite stent. It should be recognized that the pharmaceutical agent (e.g., nitric oxide or nitric oxide-releasing compound) can be present in various locations in the composite stent, including the inner layer, the outer layer, a surface of the frame layer, and/or a release layer that is located between the frame layer and the inner layer and/or the outer layer.

In a still further aspect of the inventive subject matter, the opening has an area between about 5% and 10% of a total area of the composite stent, and at least one of the openings in the frame may be covered by the inner and/or outer layer. Multiple openings can be present (up to 20, and even more), wherein at least one of the openings can be part of the terminal circumference.

Various objects, features, aspects and advantages of the present invention will become more apparent from the following detailed description of preferred embodiments of the invention.

### BRIEF DESCRIPTION OF THE DRAWING

FIG. 1 is a schematic vertical cross sectional view of a stent according to the inventive subject matter.

FIG. 2 is a schematic perspective view of a stent according to the inventive subject matter having an inner continuous layer with a layer opening.

FIG. 3 is a schematic perspective view of a stent according to the inventive subject matter having an inner and outer continuous layer with a layer opening.

### DETAILED DESCRIPTION

The inventors discovered that a stent, and especially a drug eluting stent can be fabricated in a manner that allows endothelialization of the stent in situ. Generally preferred stents include a inner and/or outer continuous layer coupled to the stent frame, wherein the stent has one or more openings extending through the frame and inner and/or outer continuous layer. The openings in contemplated stents have a size that allows endothelialization of the stent in situ.

In one preferred aspect of the inventive subject matter, a stent includes a cylindrical frame has an inner and outer surface, and a plurality of frame openings extending from the outer surface to the inner surface. A continuous inner layer having an inner layer opening is coupled to the inner surface and/or a continuous outer layer having an outer layer opening is coupled to the outer surface of contemplated stents. In such stents, the frame openings, the inner layer opening, and/or the outer layer opening coincide such that an aperture is formed that has a size sufficient to allow endothelialization. Additionally, particularly contemplated stents include a pharmaceutically active agent that is associated with the inner layer, the inner surface, a frame opening, the outer surface, and/or the outer layer.

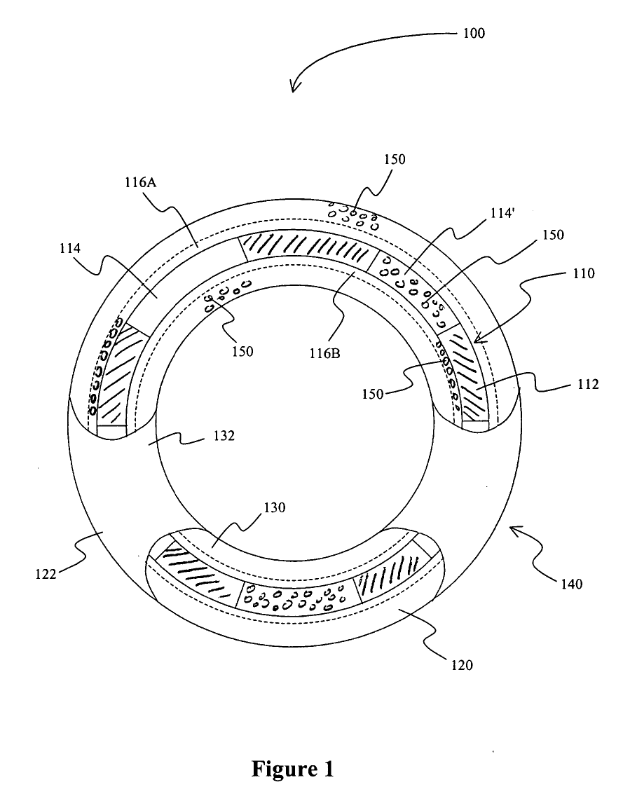

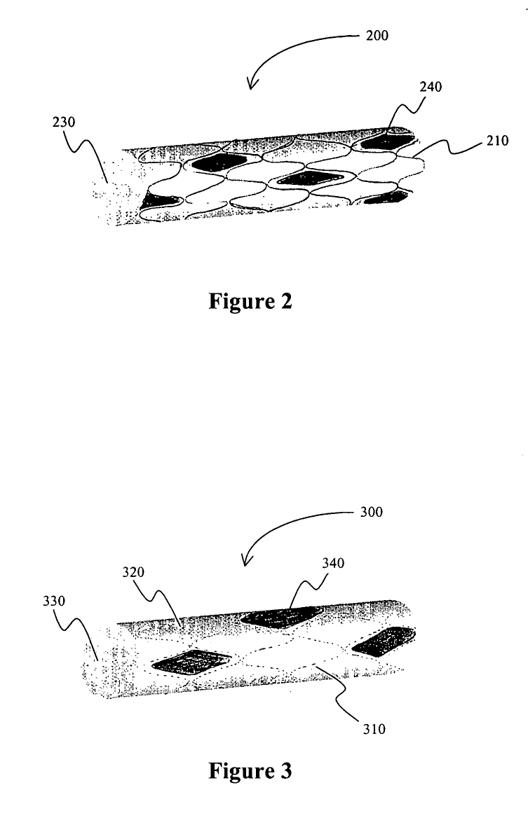

An exemplary stent according to the inventive subject matter is depicted in FIG. 1, in which stent 100 is formed by cylindrical body 110 having frame elements 112 (e.g., metal wire) and frame openings 114. Some of the frame openings may be filled with a pharmaceutically active agent 150 (optionally embedded in a matrix or other carrier) to form filled frame opening 114′. Additionally, or alternatively, the pharmaceutically active agent may also be disposed in a location other than a frame opening, and suitable alternative locations include the inside of the frame or the outside of the frame to form coating 116A and/or 116B. Once more, it should be recognized that the pharmaceutically active agent in such locations can be embedded in a matrix or other carrier, or simply be directly applied. A continuous outer layer 120 is disposed on the outside of the stent body 110 while continuous inner layer 130 is disposed on the inside of the stent body 110. Stent opening 140 extends from the inside of the stent 100 to the outside of the stent 100 such that the outer layer opening 122, the inner layer opening 132, and the frame opening 114 coincide.

With respect to the stent, it should be recognized that all known stent configurations and materials are deemed suitable so long as such stents will provide at least one, and more typically at least 2-10 openings in the cylindrical body with a size sufficient to allow endothelialization once the stent is in situ. Thus, especially contemplated stents include those for implantation to the inner wall of a blood vessel (e.g., in conjunction with percutaneous transluminal angioplasty), which may be cylindrical, semi-cylindrical, branched, etc. It should further be recognized that the material of suitable stents can vary substantially, and all known stent materials are considered suitable for use with the teachings presented herein. For example, contemplated stent materials include various metals (e.g., stainless steel, tantalum, gold, magnesium) metal alloys (e.g., nickel titanium alloy, platinum iridium, etc.), and synthetic polymers (e.g., polyethylene terephthalate, polyurethane, various acrylates, etc.). Consequently, contemplated stents may be expandable, using heat (where memory metals are used), pneumatic forces (where balloons are placed in the lumed), or other mechanisms. Alternatively, in less preferred aspects, at least a portion of the stent may be biodegradable or bioerodable in situ. Such degradation or erosion may be located in the frame body to yield a reduced frame body, or in another portion that will yield or enlarge one or more of the frame openings.

In further preferred aspects of the inventive subject matter, one or more pharmaceutically active agents are included in the stent. Particularly preferred agents will reduce or even entirely prevent smooth muscle cell proliferation, inflammation, neutrophil adhesion, platelet aggregation or other coagulation related events, and/or infections. Therefore, viewed from another perspective, suitable agents can have anti-inflammatory, anticoagulant, antifibrin, antimitotic, antithrombin, antibiotic, properties, and/or regulatory function for vascular tone, enhancement of macrophage-mediated microbial killing, and/or antioxidative properties. For example, suitable agents include actinomycin D, paclitaxel, docetaxel, heparin (e.g., low molecular weight heparin), hirudin, argatroban, forskolin, vapiprost, prostacyclin and prostacyclin analogs, dextran, dipyridamole, glycoprotein IIb/IIIa platelet membrane receptor antagonists and thrombin inhibitors. Further contemplated agents also include angiotensin converting enzyme inhibitors, calcium channel blockers, histamine antagonists, statins, antibodies and fragments thereof, phosphodiesterase inhibitors, prostaglandin inhibitors, steroids, interferons, etc.

However, particularly preferred pharmaceutically active agents include those that release nitric oxide from a precursor and/or carrier. There are numerous compositions and matters known in the art that release nitric oxide under physiological conditions, and exemplary preferred compositions include those described in U.S. Pat. Nos. 5,665,077 and 5,797,887 to Rosen, U.S. Pat. No. 6,207,855 to Toone et al., and U.S. Pat. Nos. 6,087,479 and 5,770,645 to Stamler et al., all of which are incorporated by reference herein. Thus, suitable pharmaceutically active agents include sodium nitroprusside, nitrosylated natural and synthetic polymers (e.g., polypeptides, physiologically acceptable hydrogels, etc), and other nitrosylated compounds that can release nitric oxide under physiological conditions.

With respect to the dosage or concentration of the pharmaceutically active agent in the stents according to the inventive subject matter, it is contemplated that the preferred dose is that required to produce a favorable therapeutic effect, most preferably at low or negligible toxicity and/or systemic side effects. Furthermore, it should be appreciated that the individual dosage or concentration of the pharmaceutically active agent required may also depend upon treatment specific factors (e.g., nature of the vascular injury, nature of the therapy desired, desired time of drug release, etc.). However, it should be recognized that the therapeutic effective dosage can be determined empirically, and that standard pharmacological test procedures to determine such dosages are well known to a person of ordinary skill in the art. For example, where the pharmaceutically active agent is coated onto the stent frame using a polymer, or where the inner and/or outer layer is formed from a polymer comprising the active agent, the polymer can comprise from about 1 wt % to about 40 wt %, more preferably from about 5 wt % to about 30 wt %, ad most preferably from about 10 wt % to about 20 wt %.

Thus, it should also be recognized that in contemplated stents the pharmaceutically active agent can be located in one or more locations, including the inner and outer surfaces of the stent frame, the frame opening, a channel or other depression or indentation in the stent frame, a polymeric material coupled to the stent frame or continuous outer and/or inner layer, and/or the continuous inner and/or outer layer. Depending on the type of pharmaceutically active agent, it should be recognized that the pharmaceutically active agent may be directly applied to the one or more locations (e.g., as a layer of nitrosylated polymer) or may be disposed in a pharmaceutically acceptable carrier (e.g., polyvinyl alcohol). Similarly, the pharmaceutically active agent can be evenly distributed throughout contemplated stents, or may be limited to one or more specific areas. For example, one pharmaceutically active agent (e.g., anti-clotting agent) may be disposed such that the agent is in contact with the blood flowing through the lumen on the stent, while another pharmaceutically active agent (anti-mitotic agent) may be disposed on the outside of the stent. In another example, different pharmaceutically active agents may be disposed in distinct sites (e.g., frame openings) within the stent. Thus, the pharmaceutically active agent may be administered to a site of implantation of the stent as well as systemically.

Preferred continuous inner and outer layers are preferably fabricated from a natural and/or synthetic polymer, and it is especially preferred (but not limiting to the inventive subject matter) that contemplated stents will include both a continuous inner and an outer layer. As used herein, the term “continuous inner layer” refers to a configuration of a material in which the material forms a curved surface over the inner surface of the stent frame and wherein the curved surface has a radius that is substantially identical with the radius of the inner surface of the stent frame (less than 20% absolute difference). Similarly, the term “continuous outer layer” refers to a configuration of a material in which the material forms a curved surface over the outer surface of the stent frame, wherein the curved surface has a radius that is substantially identical with the radius of the outer surface of the stent frame (less than 20% absolute difference). Viewed from another perspective, the continuous inner and/or outer layer are direct or indirect coatings of the stent frame, wherein the coatings have at least one opening that coincides with a frame opening. Thus, a coated wire of a wire stent will not fall within the scope of the definition provided above.

It should be appreciated that the continuous outer and inner layer may be coupled to the cylindrical stent frame in numerous manners, and all known manners of coupling are deemed suitable for use herein. For example, where the continuous outer and inner layer comprise a prefabricated material, such material may be present in cylindrical shape having a radius that allows mating engagement of the cylinders with the cylindrical stent frame. The layers may then be affixed to the stent frame using an adhesive. Alternatively, the adhesive may be replaced or complemented with a polymeric material that includes the pharmaceutically active agent. On the other hand, at least one of the continuous outer and inner layers may also be formed on the surface (or polymeric coat that is disposed on the surface) of the stent frame. In such event, it should be recognized that all manners of coating are deemed suitable for use herein and include spray coating, dip-coating, etc.

Consequently, contemplated continuous outer and inner layers may be fabricated from numerous materials and combinations thereof, and particularly suitable materials include various polymers (e.g., polyesters, polyamides, polyacrylates, etc.), which may or may not be fluorinated. Depending on the particular configuration of the stents, the material may vary considerably. For example, where a pharmaceutical compound is sodium nitroprusside to release nitric oxide as the pharmaceutically active agent, especially preferred outer and inner layers comprise a fluorinated polymer, and particularly expanded polytetrafluoroethylene. Viewed from another perspective, it is generally preferred that the continuous inner and/or outer layer will include a material having selective permeability with respect to nitric oxide. For example, expanded polytetrafluorethylene (ePTFE) can be produced with controlled pore size, and the nitric oxide donor molecule can be retained in the stent while the nitric oxide is released from the layer (most preferably at a predetermined rate). On the other hand, where nitric oxide is released from a synthetic polymeric matrix, the suitable polymer may be a hydrogel. Furthermore, it should be recognized that additional therapeutic materials can be deposited in the stent openings or between the first and second layers. As will be understood by a person of ordinary skill in the art, at least one of the continuous inner and outer layers is then permeable or selectively permeable to the additional therapeutic materials. Regardless of the particular choice of material, it is generally preferred that the continuous outer and inner layers have a composition that allows radial expansion of the stent once the stent is in situ. Thus, any graft material known in the art is also contemplated herein.

With respect to the openings in contemplated stents, it should be recognized that all known manners of creating an opening in the materials of the stent are deemed appropriate, including cutting, laser ablation, and sanding. For example, where the continuous inner and outer layers are prefabricated and arranged to enclose the stent frame, laser light or other energy is preferably applied to create the opening through inner and outer layers. Alternatively, in less preferred aspects, the openings can be created sequentially (e.g., by manual cutting). Regardless of the manner of creating the opening, it should be recognized that the opening will, in all contemplated stents extend through the frame (via a frame opening) and the continuous inner and/or outer layer.

In especially preferred aspects, where high energy is applied to create the opening, it is contemplated that part of the energy is employed to seal the opening. For example, the inner and/or outer layer can be annealed to the frame at the position of the frame opening, and/or the inner and outer layer may be annealed together (optionally also including any intervening layers). Thus, it should be recognized that the step of forming an opening can be used to form a seal that will assist in retaining one or more pharmaceutically active agents in a space defined between the stent frame and a layer surrounding the stent frame.

Typically, the size of contemplated openings will be substantially homogeneous (typically within ⁺/₋10% absolute), and it is further contemplated that the opening will be not less than 30%, more typically no less than 50%, and most typically no less than 70% of the size of the corresponding frame opening. Furthermore, contemplated stents include at least one opening that has a size to allow endothelialization. Therefore, it is generally contemplated that the smallest dimension of the opening is at least 100 micrometesr, more typically 300 micrometers, even more typically at least 500 micrometers, and most typically at least 1 millimeter. With respect to the largest dimension, it is generally contemplated that the largest dimension of the opening is at least 500 micrometers, more typically 1 millimeter, even more typically at least 3 millimeters, and most typically at least 5 millimeters. Typically, the area of contemplated stent openings will be between about 1 and 75%, and more typically between about 2 and 25% of the entire surface area of the stent. Thus, the number of openings will typically be between about 1 and 100, and more typically between 1 and 20. Furthermore, the frame openings are preferably entirely formed within the stent surface. However, in alternative aspects, one or more openings may also coincide with the front- and/or back end of the stent (terminal circumference). In less preferred aspects, the one or more of the frame openings may also be covered by one or more of the continuous outer and inner layers.

Thus, viewed from another perspective, it should therefore be appreciated that a composite stent has a cylindrical multilayer structure comprising an inner layer at least partially surrounded by a frame layer that is at least partially enclosed by an outer layer, and further comprises an opening extending through the multilayer structure, wherein the opening has a size sufficient to allow endothelialization when the composite stent in implanted into a blood vessel.

It should further be appreciated that the stents according to the inventive subject matter may also be employed in numerous tissues other than a blood vessel, and alternative sites include the airway, the gastrointestinal tract, the bladder, the uterus, and the corpus cavernosum. Thus, the compositions, methods and devices of the present invention can be use to treat respiratory disorders, gastrointestinal disorders, urological dysfunction, impotence, uterine dysfunction, and premature labor. Nitric oxide delivery at a treatment site can also result in smooth muscle relaxation to facilitate insertion of a medical device, for example in procedures such as bronchoscopy, endoscopy, laparoscopy and cystoscopy. Alternatively, or additionally, delivery of NO can also be used to prevent cerebral vasospasms post hemorrhage and to treat bladder irritability, urethral strictures and biliary spasms.

### EXAMPLES

In one exemplary aspect of the inventive subject matter, the stent is a meshed metal wire stent with a plurality of frame openings. Most preferred stents include those for insertion into a coronary artery and are sized and configured accordingly. There are numerous such stents known in the art, and all such stents are deemed suitable for use herein. A pharmaceutically active agent (preferably sodium nitroprusside or substituted nitroso compound in a pharmacologically acceptable polymer) is coated onto the outer surface of the stent by dip-coating, and the active agent may therefore be present on the inner and outer surface of the stent as well as in at least some of the stent openings. Either or both of the inner and outer continuous layers can be preformed for subsequent coupling to the stent frame or be formed directly on the stent frame. Furthermore, additional (polymeric) layers can be added to cover the stent and/or pharmaceutically active agent.

In a first exemplary manner of manufacture, the stent frame is immersed in a liquid that includes a pharmaceutically active agent to fill the stent frame openings and to form a coat on at least one of the inner and outer surfaces of the stent frame. The liquid coating material is then dried or cured to solidify the carrier and fix the pharmaceutically active agent on the stent frame surface and/or in stent frame openings. After drying, the inner continuous layer and/or outer continuous layer is formed by immersion coating onto the coated stent frame. The openings may then be formed as described below.

In a second exemplary manner of manufacture, the stent frame is optionally immersed in a liquid that includes a pharmaceutically active agent to fill the stent frame openings and to form a coat on at least one of the inner and outer surfaces of the stent frame. Then, the optionally coated stent is placed between preformed, tubular, inner and outer continuous layers, which can be sealed at one end of the stent to create a sealed space between the optionally coated stent surface and the tubular inner and outer layers. Alternatively, coupling can preferably be performed by gluing or annealing the inner/outer continuous layer to the optionally coated frame. A pharmaceutically active agent can be injected into the sealed space, and the second end can be sealed, wherein the pharmaceutically active agent can be dried or cured prior to sealing the second end of the stent. The openings may then be formed as described below.

Openings are preferably formed by cutting or laser ablation to achieve a complete channel that extends from the lumen of the stent to the outer surface of the final stent. Most preferably, where the openings are thermally created, sufficient heat energy is provided to not only form the channels, but also to seal the outer continuous layer to the inner continuous layer at the inside of the channel. Such fusion advantageously contains the pharmaceutically active agent to prevent inadvertent release of the pharmaceutically active agent to the patient. Where additional layers are present (e.g., layer comprising or covering the pharmaceutically active agent, wherein that layer may be at least partially surrounding the outer and/or inner stent surface), it should be recognized that thermal generation of the opening may seal at least one of the additional layers to at least one of the inner and outer continuous layers.

Thus, specific embodiments and applications of stents covered by a layer having a layer opening have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. A stent, comprising:
a cylindrical frame having an outer surface, an inner surface, and a plurality of frame openings extending from the outer surface to the inner surface;
at least one of a continuous inner layer coupled to the inner surface and having an inner layer opening, and a continuous outer layer coupled to the outer surface and having an outer layer opening;
wherein at least one of the frame openings, and at least one of the inner layer opening and the outer layer opening coincide such that an aperture is formed having a size sufficient to allow endothelialization, and
wherein a pharmaceutically active agent is associated with at least one of the inner layer, the inner surface, another one of the frame openings, the outer surface, and the outer layer.

2. The stent of claim 1 comprising the continuous inner layer and the continuous outer layer.

3. The stent of claim 1 wherein the pharmaceutically active agent is disposed onto the inner surface and the outer surface.

4. The stent of claim 1 wherein the pharmaceutically active agent is disposed within the another one of the frame openings.

5. The stent of claim 1 wherein the pharmaceutically active agent is covered by the inner layer.

6. The stent of claim 1 wherein the pharmaceutically active agent is covered by the outer layer.

7. The stent of claim 1 wherein the pharmaceutically active agent is disposed in a polymeric matrix that is coated onto the cylindrical frame and at least partially covered by the continuous outer layer.

8. The stent of claim 1 wherein the pharmaceutically active agent has an activity selected from the group consisting of modulation of vascular tone, inhibition of neutrophil adhesion, enhancement of macrophage-mediated microbial killing, inhibition of platelet adhesion, inhibition of platelet aggregation, and inhibition of smooth muscle cell proliferation.

9. The stent of claim 1 wherein at least one of the continuous inner and outer layers is fabricated from a material that allows diffusion of a gas and that prevents diffusion of a liquid.

10. The stent of claim 1 wherein at least one of the continuous inner and outer layers is fabricated from a fluorinated synthetic polymer.

11. The stent of claim 1 wherein the cylindrical frame, the inner layer, and the outer layer are fabricated from a material that allows radial expansion of the stent.

12. The stent of claim 1 wherein the cylindrical frame comprises a metal.

13. A composite stent having a cylindrical multilayer structure comprising an inner layer at least partially surrounded by a frame layer that is at least partially enclosed by an outer layer, and further comprising an opening extending through the multilayer structure, wherein the opening has a size sufficient to allow endothelialization when the composite stent is implanted into a blood vessel.

14. The composite stent of claim 13 further comprising a pharmaceutical agent that provides a pharmaceutically active compound to a site of implantation of the composite stent.

15. The composite stent of claim 14 wherein the pharmaceutical agent is present in a location selected from the group consisting of the inner layer, the outer layer, a surface of the frame layer, and a release layer that is located between the frame layer and at least one of the inner layer and the outer layer.

16. The composite stent of claim 13 wherein the pharmaceutically active compound is nitric oxide.

17. The composite stent of claim 13 wherein the opening has an area between about 5% and 25% of a total area of the composite stent.

18. The composite stent of claim 13 further comprising a frame layer opening that is at least partially covered by at least one of the outer layer and the inner layer.

19. The composite stent of claim 13 further comprising between one and twenty additional openings that have a size sufficient to allow endothelialization when the composite stent in implanted into the blood vessel.

20. The composite stent of claim 13 wherein the opening is part of a terminal circumference of the stent.
